(19) United States
(12) Patent Application Publication
(10) Pub. No.: US 2004/0037774 A1
(43) Pub. Date: Feb. 26, 2004

(54) Carrier for carrying radioactive substances and methods for using the same

(76) Inventor: Chuang - Chun Chiueh, Taipei (TW)

Correspondence Address: BACON & THOMAS, PLLC, 625 SLATERS LANE, FOURTH FLOOR, ALEXANDRIA, VA, 22314

(21) Appl. No.: 10/291,382

(22) Filed: Nov. 12, 2002

(30) Foreign Application Priority Data
Aug. 26, 2002 (TW) 91119232

(51) Int. Cl. A61K 51/00

(52) U.S. Cl. 424/1.69

(57) ABSTRACT

The invention provides a carrier for carrying radioactive substances and methods for using the same, wherein phycoerythrin is served as a carrier for carrying radioactive substances so as to enable accumulation of radioactive substances specifically in the tumor cells and the radiation emitted therefrom kills the tumor cells effectively. Therefore, the invention can be applied in the treatments for various types of cancers.

### BACKGROUND OF THE INVENTION

1. Field of the Invention

The invention provides a carrier for carrying radioactive substances and methods for using the same, wherein phycoerythrin is served as a carrier for carrying radioactive substances so as to enable accumulation of radioactive substances specifically in the tumor cells and the radiation emitted by the radioactive substances leads to killing of tumor cells effectively.

2. Description of Related Arts

Cancer has been the number one life-threatening risk factor for nearly one hundred years. In the United States, one-third of the population is estimated to suffer from cancer, and one-fifth of the total deaths are attributed to cancer. In Taiwan, cancer has ranked the first of the ten leading causes of death since 1982. The current treatments for cancer, aiming at damaging or inhibiting proliferation of tumor cells, are surgery, radiation therapy, chemotherapy, or combinations thereof.

Radiation therapy, which utilizes radioactive substances for cancer treatment, has been practiced for nearly twenty years. Cells with rapid division cycles, such as tumor cells, are particularly susceptible to radiation and undergo metabolism disorder and death ultimately when they are exposed to sufficient amount of radiation. To date, the best example of radioactive substances used in cancer treatment is radioactive iodine for treating thyroid cancer. Radioactive iodine can be delivered simply by oral route. Due to the unique ability of thyroid tissue to concentrate iodine, the radioactive iodine is retained effectively inside the thyroid gland, and cancerous thyroid tissue also retains the ability of radioactive iodine uptake. By the beta radiation emitted from radioactive iodine the cancerous thyroid tissue is destroyed and thus achieve the purposes of elimination, prevention of recurrence and metastasis.

Furthermore, the range of beta radiation emitted by radioactive iodine in cancerous thyroid tissue is about 2 mm, hence the damage to normal, adjacent tissues is minimized.

However the current radiation therapy is not truly selective, i.e., it kills both tumor cells and normal tissues as well. For example, the use of cobalt-60 in external bean radiation therapy, in which the radiation passes through normal tissues to tumor cells and is thus more destructive to normal, adjacent tissues.

Radioactive iodine reaches specific therapeutic effect due to the unique ability of thyroid tissue to concentrate iodine, however, radioactive iodine can not be concentrated by other kinds of tumor cells, and thus is not suitable for curing other kinds of cancers. Implantation of radioactive substances into specific tissues can be achieved through surgical procedures, nevertheless, the surgery do bring harm to human body.

According to the foregoing drawbacks and advantages of radioactive materials, a carrier for carrying radioactive substances to specific tumor tissue is in considerable need.

### SUMMARY OF THE INVENTION

For overcoming the foregoing drawbacks of the prior arts, the present invention provides a carrier for carrying radioactive substances, wherein phycoerythrin (PE) is served as a carrier for carrying radioactive substances so as to enable accumulation of radioactive substances specifically in the tumor cells.

Another objective of the invention is to provide a method for introducing radioactive substances into tumor cells by using phycoerythrin as a carrier. The method comprises the steps of providing phycoerythrin; conjugating phycoerythrin with the radioactive substances to be carried; introducing the radioactive substances-carried phycoerythrin into tumor cells; accumulating the foregoing radioactive substances in tumor cells; and killing tumor cells by the radiation emitted from the radioactive substances.

The foregoing phycoerythrin can be R-phycoerythrin (R-PE) or B-phycoerythrin (B-PE), wherein R-phycoerythrin is more preferable.

The foregoing phycoerythrin that accumulates in tumor cells is able to facilitate determination of tumor localization and range. The method for using the same comprises: introducing effective amount of phycoerythrin into tumor cells; exposing phycoerythrin to specific light source so as to emit fluorescence at specific wavelength; and determining the localization and range of tumor.

The absorption wavelength of the foregoing R-phycoerythrin is at the range from 488 nm to 633 nnm, wherein the ideal wavelength is at 488 nm, and the emission wavelength of red fluorescence is at 575 nm. The absorption wavelength of the foregoing B-phycoerythrin is at the wavelength range from 488nm to 633nnm, wherein the ideal wavelength is at 543 nm, and the emission wavelength of red fluorescence is at 575 nm.

The foregoing radioactive substances comprise any of which possesses tumor-killing ability and can easily conjugate with proteins, for example, radioactive iodine, which can emit beta radiation to kill tumor cells.

### BRIEF DESCRIPTION OF DRAWINGS

FIG. 1 shows the fluorescence spectrum of the R-phycoerythrin.

FIG. 2 shows the fluorescence spectrum of the B-phycoerythrin.

FIG. 3 shows that R-phycoerythrin does not accumulate in human fibroblast cells (normal cells).

FIG. 4 shows that R-phycoerythrin apparently accumulates in human neuroblastoma cells (tumor cells).

### DETAILED DESCRIPTION OF THE PREFERRED EMBODIMENTS

The invention provides a carrier for carrying radioactive substances, wherein phycoerythrin is served as a carrier for carrying radioactive substances and enables radioactive substances to accumulate specifically in the tumor cells. According to the results of this invention, phycoerythrin, which exists exclusively in Microalgae, has high affinity to tumor cells and selectively accumulates therein while does not stay in normal cells. Therefore, the present invention applies the special feature of phycoerythrin to cancer treatment by using phycoerythrin as a carrier for carrying radioactive substances.

Another objective of the invention is to provide a method for introducing radioactive substances into and killing of tumor cells by using phycoerythrin as a carrier, comprising the steps of providing phycoerythrin; conjugating phycoerythrin with the radioactive substances to be carried; introducing the radioactive substances-carried phycoerythrin into tumor cells; accumulating the radioactive substances in forgoing tumor cells; and killing tumor cells by the radiation emitted from radioactive substances carried by phycoerythrin.

Iodine, an example of general radioactive substances, possesses the ability to react with proteins and can bind to tyrosine efficiently, and is also one of the common radioactive elements for medical use. Accordingly, one example of the present invention chooses radioactive iodine to be carried by phycoerythrin and be introduced to the tumor cells . By using the ability of beta radiation emission of radioactive iodine, the tumor cells are killed efficiently and specifically without damaging normal cells.

Furthermore, the foregoing phycoerythrin can be introduced into tumor cells in an effective amount. And by irradiating with specific light source, phycoerythrin is induced to emit fluorescence at a specific wavelength so that the localization and range of tumor can be determined.

The foregoing phycoerythrin can be R-phycoerythrin (R-PE) or B-phycoerythrin (B-PE), wherein the R-phycoerythrin is more preferable.

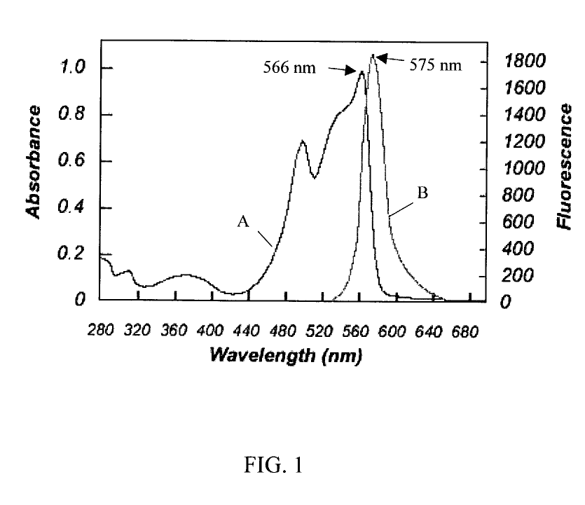

R-phycoerythrin is a water-soluble, fluorescent pigment protein existed exclusively in algae. The major physical and chemical properties of R-phycoerythrin are as follows: Its structure consists of three types of subunits, α-β and γ, which usually appear in hexamer form of (αβ)_{6γ}. The molecular weight of R-phycoerythrin is 260,000 daltons. The absorption spectrum, as in FIG. 1A, shows the primary absorbance peak at 566 nm with a secondary peak at 498 nm. The fluorescence emission spectrum, as in FIG. 1B, shows the peak of the fluorescence wavelength is at 575 nm with the maximum excitation wavelength being 488 nm, at which the fluorescence quantum yield is 0.84.

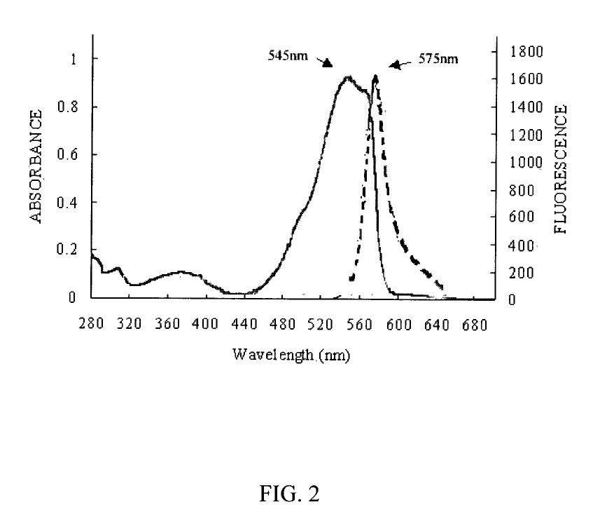

The major physical and chemical properties of B-phycoerythrin are as follows: Its structure consists of three types of subunits, α-β and γ, which usually appear in hexamer form of (αβ)_{6γ}. The molecular weight of R-phycoerythrin is 240,000 daltons. The range of absorption wavelength lies within the visible light. The absorption spectrum, as in FIG. 2 (solid line), shows the primary absorbance peak at 545 nm with a secondary peak at 498 nm. The range of fluorescence emission wavelength lies within the visible light. The fluorescence emission spectrum, as in FIG. 2 (dashed line), shows the peak fluorescence wavelength is at 575 nm. The fluorescence is strong and stable with little influenced by environmental pH value, temperature and existence of other biomoleculars. The maximum excitation wavelength is 543 nm, at which the fluorescence quantum yield is 0.98.

The term ‘tumor cells’ used herein refers to all types of tumor cells without specific limitation, and the doses of phycoerythrin to be treated can vary depending on the type of tumor cells.

The following is a detailed description of the best presently known modes of carrying out the inventions. This description is not to be taken in a limiting sense, but is made merely for the purpose of further illustrating the present inventions.

Best Mode of Present Invention

R-phycoerythrin can Selectively Accumulate in Tumor Cells

(1) Preparation of R-phycoerythrin:

The specification information of R-phycoerythrin is as follows: 1. The absorbance ratio: A₅₆₆/A₂₈₀≧5.3; A₅₆₆/A₄₉₈≦1.5; A₆₂₀/A₅₆₆≦0.005. 2. The purity of the protein is greater than 98%. 3. It is stored in 150 mM sodium phosphate buffer (pH 7.0) containing 60% saturated ammonium sulfate, 1 mM EDTA (Sodium Ethylene-diaminetetraacetate), and 1 mM sodium azide, and needs to be stored at 4° C.

R-phycoerythrin is complete dialyzed with 150 mM sodium phosphate buffer, and then serial dilution is carried out by using MEM medium as diluent to obtain the R-phycoerythrin dilutions of concentrations between 1-100 μg/ml.

(2) Preparation of Tumor Cells

The tumor cells are human neuroblastoma cells, and normal cells for comparison are human fibroblast cells. First, cells are maintained in MEM medium (pH 7.4) supplemented with 10% fetal bovine serum, and incubate at 37° C. and 95% humidity. Replace the MEM medium every two days. When carrying out cell subculture, first remove the medium, and rinse the cells once with sodium phosphate buffer. Then add 0.25% trypsin-EDTA to detach the cells from the surface of petri dish, and stop the reaction of trypsin-EDTA by giving proper amount of foregoing MEM medium, and thus the cell suspension is obtained. Secondly, seed 100,000 to 400,000 cells onto cover slips placed in a 12-well culture plate and incubate at 37° C. and 95% humidity for 16 hours.

(3) Affinity Test on R-phycoerythrin for Normal and Tumor Cells

Following is the affinity test on R-phycoerythrin for human fibroblast cells (normal cells) and human neuroblastoma cells (tumor cells). Take the 12-well culture plate with cells growing on cover slips mentioned in step 2. Remove the medium and rinse the cells once with sodium phosphate buffer. Add 1 mL of R-phycoerythrin dilutions mentioned in step 1 to the cells and follow with incubation at 37° C. and 95% humidity. After 24-hour incubation, remove the medium and rinse the cells three times with sodium phosphate buffer. Then add 4% of paraformaldehyde to each well of the culture plate and incubate at room temperature until the cells are completely fixed on cover slips. After washing once with sodium phosphate buffer, pick the cover slip up from the culture plate and embed the cells with 80% glycerol. Observe the staining by fluorescent microscopy.

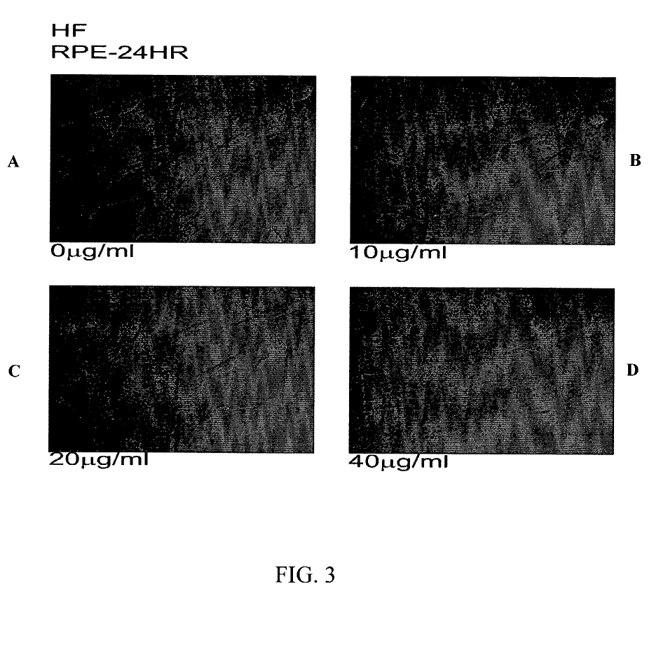

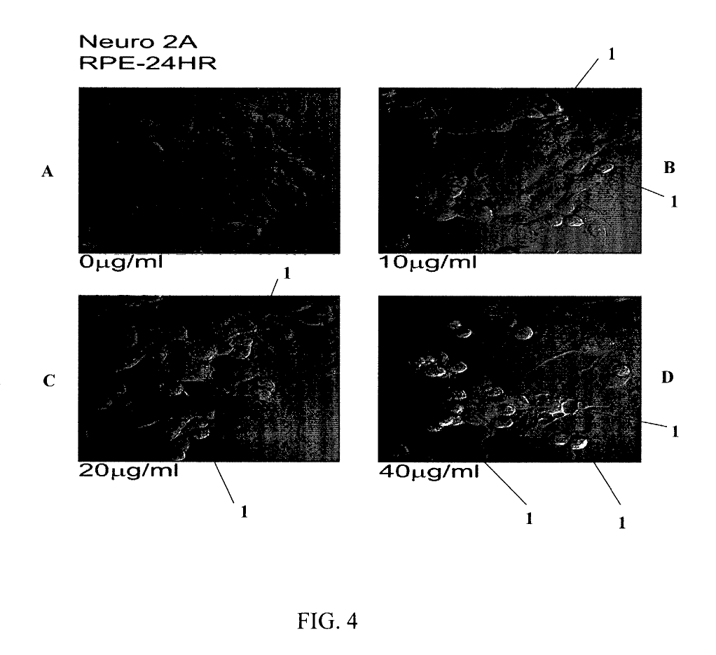

The results are shown in FIG. 3 and FIG. 4. FIG. 3A shows fluorescence image of normal human fibroblast cells without adding R-phycoerythrin, while FIG. 3B, 3C, and 3D show that of normal human fibroblast cells added with 10 μg/ml, 20 μg/ml, 40 μg/ml of R-phycoerythrin, respectively. The results show that no fluorescent signal is observed in human fibroblast cells, indicating that R-phycoerythrin does not accumulate in normal cells.

FIG. 4A shows the fluorescence image of human neuroblastoma cells without adding R-phycoerythrin, while FIG. 4B, 4C, and 4D show that of human neuroblastoma cells added with 10 μg/ml, 20 μg/ml, 40 μg/ml of R-phycoerythrin, respectively. The results show that the strength of fluorescence signal in human neuroblastoma cells is in accordance with the increasing amount of R-phycoerythrin (see the bright area 1 as indicated in FIG. 4), suggesting that R-phycoerythrin selectively retains in tumor cells.

In conclusions, the present invention utilizes R-phycoerythrin in cancer radiation therapy as a carrier that effectively carries radioactive substances. By introducing effective amount of R-phycoerythrin into tumor cells as described in the best mode, the result demonstrates that: 1. R-phycoerythrin can selectively accumulated within tumor tissues. 2. When being exposed to specific light source (at the wavelength ranging from 488 nm to 633 nm, wherein the ideal is at 488 nm), R-phycoerythrin emits red fluorescence (at the wavelength of 575 nm), which facilitates determination the localization and range of tumor. 3. By introducing R-phycoerythrin conjugated in advance with radioactive iodine enables killing of tumor cells via the beta radiation emitted from radioactive iodine.

What is claimed is:

1. A carrier for carrying radioactive substances, wherein phycoerythrin (PE) is served as said carrier for carrying radioactive substances and enables radioactive substances to accumulate specifically in tumor cells.

2. The carrier as in claim 1, wherein said phycoerythrin can be R-phycoerythrin (R-PE) or B-phycoerythrin (B-PE).

3. The carrier as in claim 2, wherein said carrier is preferably R-phycoerythrin.

4. The carrier as in claim 1, wherein said phycoerythrin that accumulates in tumor cells can facilitate determination of the localization and range of tumor.

5. The carrier as in claim 2, wherein the range of the absorption wavelength of said R-phycoerythrin is from 488 nm to 633 nnm.

6. The carrier as in claim 5, wherein said wavelength is preferably at 488 nm.

7. The carrier as in claim 2, wherein the range of the absorption wavelength of said B-phycoerythrin is from 488 nm to 633 nnm.

8. The carrier as in claim 7, wherein said wavelength is preferably at 543 nm.

9. The carrier as in claim 1, wherein said radioactive substances comprise any of which possesses tumor-killing ability and can easily conjugate with proteins.

10. The carrier as in claim 9, wherein said radioactive substances can be radioactive iodine.

11. A method for introducing radioactive substances into tumor cells by using phycoerythrin as a carrier, which comprises:
providing phycoerythrin;
conjugating phycoerythrin with the radioactive substances to be carried;
introducing the radioactive substances-carried phycoerythrin into tumor cells;
accumulating the radioactive substances in said tumor cells; and
killing tumor cells by the radiation emitted from radioactive substances carried by phycoerythrin.

12. The method as in claim 11, wherein said phycoerythrin can be R-phycoerythrin (R-PE) or B-phycoerythrin (B-PE).

13. The method as in claim 12, wherein said carrier is preferably R-phycoerythrin.

14. The method as in claim 13, wherein the range of the absorption wavelength of said R-phycoerythrin is from 488 nm to 633 nnm.

15. The method as in claim 14, wherein said wavelength is preferably at 488 nm.

16. The method as in claim 11, wherein said phycoerythrin that accumulates in tumor cells can facilitate determination of the localization and range of tumor.

17. The carrier as in claim 11, wherein said radioactive substances can be radioactive iodine.
